(19) United States
(12) Patent Application Publication
(10) Pub. No.: US 2019/0183723 A1
(43) Pub. Date: Jun. 20, 2019

(54) ABDOMINAL MASSAGING SYSTEM FOR IMPROVING GASTRIC REFLUX, TREATING HIATAL HERNIAS AND REDUCING ABDOMINAL TENSION

(71) Applicant: Ryan Vincent SWENSON, Rapid City, SD (US)

(72) Inventor: Ryan Vincent SWENSON, Rapid City, SD (US)

(21) Appl. No.: 16/175,440

(22) Filed: Oct. 30, 2018

Related U.S. Application Data
(60) Provisional application No. 62/598,874, filed on Dec. 14, 2017.

(51) Int. Cl. A61H 15/02; A61H 15/00; A61H 23/02; A61H 7/00

(52) U.S. Cl. CPC A61H 15/02; A61H 15/0078; A61H 23/0254; A61F 7/00; A61H 2015/0021; A61H 2201/0207; A61H 2201/10; A61H 7/007

(57) ABSTRACT

A wearable system for improving gastric reflux, treating hiatal hernias or reducing abdominal tension using an abdominal massaging device that has a flexible wall membrane that is positioned against a user's chest immediately below the user's sternum with internal rotating arms in the device pushing downwardly on the chest one after another with downwardly, outwardly tapering top edges of the device resting against the bottom of the user's rib cage below the user's sternum to resist any upward movement of the device.

### RELATED APPLICATION

The present application claims priority to U. S. Provisional Patent Application Ser. No. 62/598,874, entitled Mechanical Abdominal Massager For Improving Gastric Reflux, Hiatal Hernias And Abdominal Tension, filed Dec. 14, 2017, incorporated herein by reference in its entirety for all purposes.

### TECHNICAL FIELD

The present system relates to therapeutic massaging devices.

### BACKGROUND OF THE INVENTION

Gastric reflux, hiatal hernias and abdominal tension are common maladies in our world. There is a need for natural methods to help these conditions. Unfortunately, previous approaches to solving these problems relied on medications. Unfortunately, medicated approaches tend to only address symptoms instead of the underlying causes of the problem. In addition, medications too often come with unwanted side-effects. As a result, people tend to avoid using medication until their condition becomes quite severe. This may lead to an unmanageable situation requiring surgical intervention. Other approaches, such as deep breathing, yoga, etc. require active participation and effort by the subject, and people tend to avoid (or not take time) for them as well.

What is instead desired is a simple mechanical solution to these problems that can be performed on the go (without taking time away from the user's busy daily schedule). Preferably, the therapeutic solution to these problems would require no active participation by the user other than putting on a small, comfortable, wearable device. As will be shown, the present system provides such a solution.

### SUMMARY OF THE INVENTION

The present system provides an abdominal massaging assembly, comprising: a housing with an internal cavity therein; a flexible wall membrane disposed on one side of the housing; an elastic support member connected to the housing for supporting the housing such that the flexible wall membrane is positioned against a user's chest immediately below the user's sternum; and a plurality of rotating massage arms disposed within the internal cavity of the housing, wherein the rotating arms sequentially push the flexible wall membrane against the user's chest, and wherein the rotating arms all sequentially move in a downwardly direction against the flexible wall membrane. Preferably, each of the plurality of rotating massage arms has a roller at its distal end which rolls against the flexible wall membrane.

In preferred embodiments, the top of the housing comprises a pair of downwardly, outwardly tapering edges dimensioned to rest against the bottom of the user's rib cage and below the user's sternum. These downwardly, outwardly tapering edges push against the bottom of the user's rib cage and sternum as the rotating arms (moving in the downwardly direction against the flexible wall membrane) push the housing upwards on the user's chest. As such, due to its unique shape, the present device remains centered at the optimal position on the user's chest at the bottom of the user's sternum adjacent to the xiphoid process of the sternum.

In preferred embodiments, the housing also includes a motor and battery for rotating the plurality of arms, and a control system for controlling the speed of movement of the arms. Therapeutic heating and vibration elements may optionally be added as well.

As such, the present system also provides a novel method of improving gastric reflux, treating hiatal hernias and reducing abdominal tension by: placing a flexible wall membrane of an abdominal massaging device against a user's chest immediately below the user's sternum, and then rotating the massage arms in the housing such that the arms sequentially push against the flexible wall membrane moving in a downwards direction against the user's chest.

Advantages of the present mechanical system include that it has no medication side-effects and operates in a passive therapy manner, requiring no active participation by the user except for putting on a small wearable device. The present system is natural, discreet, mobile and inexpensive.

### BRIEF DESCRIPTION OF THE DRAWINGS

FIG. 1 is a front elevation view of the present massaging device.

FIG. 2 is a side elevation view of the present massaging device.

FIG. 3 is a front elevation view of the present massaging device with its top cover removed showing internal components of the system.

FIG. 4 is a side elevation view of the present massaging device with the top cover removed showing internal components of the system.

FIG. 5 is a view showing a patient wearing the present massaging device.

### DETAILED DESCRIPTION OF THE DRAWINGS

FIGS. 1 and 2 show the present abdominal massaging device 30 comprising a hard shelled housing 25 (formed from top shell 13 and bottom shell 10) defining an internal cavity therein. The bottom shell 10 faces against the patient's chest. A flexible wall membrane 22 is disposed across a portion of bottom shell 10. As seen in FIG. 5, an elastic support member 21 that wraps around the patient is connected to housing 25 for supporting housing 25 such that flexible wall membrane 22 is positioned against a user's chest immediately below the user's sternum. In preferred aspects, flexible membrane 22 could be made of rubber, be silicone-based or made of some other material. Flexible membrane 22 may be placed either directly against the user's skin or against the user's clothing. As seen in FIG. 1, a detachable buckle 18 can be used to secure elastic support member 21 onto housing 25 after elastic support member 21 has been wrapped around the user's torso (as seen in FIG. 5). Preferably, housing 25 is waterproof as well.

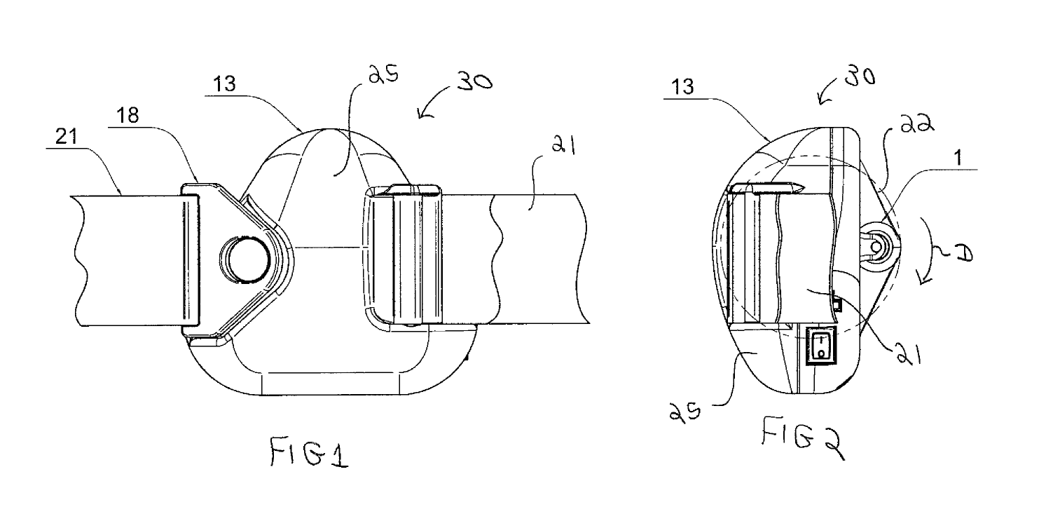

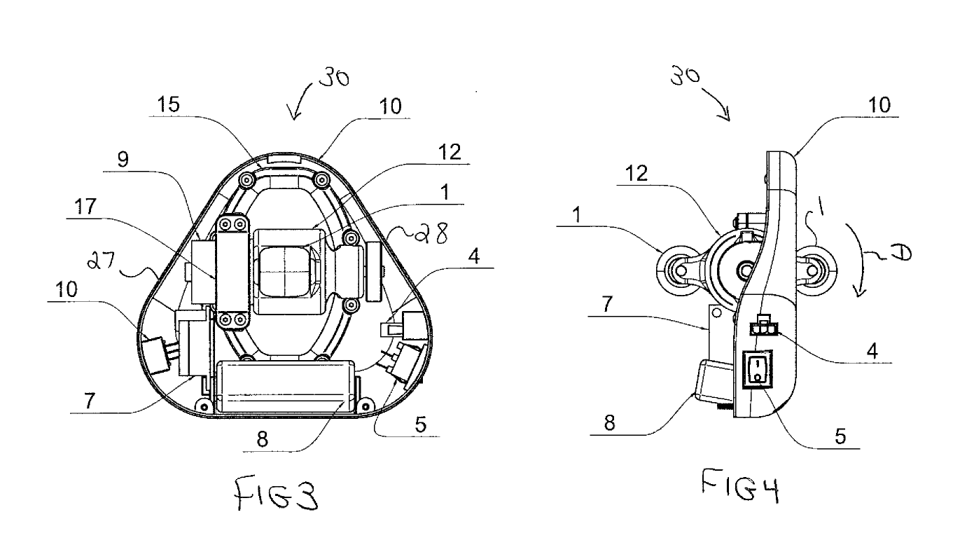

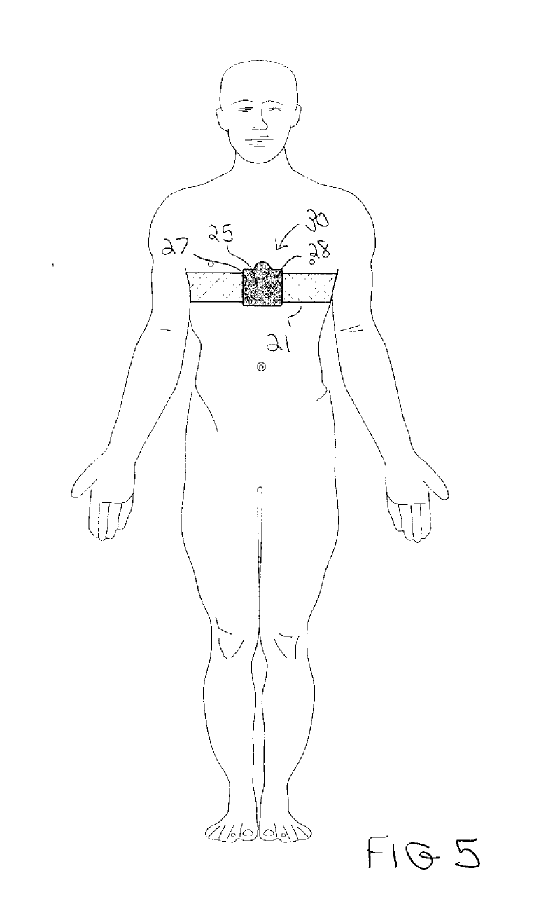

As best seen in FIGS. 2 and 4, a plurality of rotating massage arms 1 are disposed within housing 25, and rotating arms 1 sequentially push flexible wall membrane 22 against the user's chest. Moreover, rotating arms 1 each sequentially move in a downwardly direction D against flexible wall membrane 22. As a result, the user's chest is massaged with a downwardly sweeping motion (i.e.: with arms 1 travelling downwards one after another on the user's chest).

As best seen in FIGS. 3 and 5, the top of the housing 25 comprises a pair of downwardly, outwardly tapering edges 27 and 28 dimensioned to rest against the bottom of the user's rib cage below the user's sternum adjacent to the sternum's xiophod process. In operation, the downwardly, outwardly tapering edges 27 and 28 push against the bottom of the user's rib cage and sternum as the rotating arms 1 move in downward direction D against flexible wall 22. This novel housing shape has the advantage of preventing the device from walking upwardly on the user's chest (while massaging the user in a continuous downward direction). Specifically, as best seen in FIGS. 3 and 5, housing 25 has a generally triangular shape.

As best seen in FIG. 2, each of the plurality of rotating massage arms 1 has a roller at its distal end, and the rollers roll against inside surface of flexible wall membrane 22 as they move downwardly in direction D. Preferably, as best seen in FIG. 4, rotating massage arms 1 preferably extend from a rotating axle 12 mounted within the internal cavity of housing 25. As can be seen, rotating axle 12 is preferably positioned parallel to elastic support member 21 (i.e.: rotating axle 12 and elastic support member 21 are both positioned horizontally and parallel to the ground).

Additional interior components of housing 25 are best seen in FIGS. 3 and 4, as follows. A motor 9 is provided to rotate axle 12 which in turn moves rotating arms 1. Battery pack 8 provides power to motor 9. A clamp 17 holds rotating axle 12 in position. A charge connector 4 can be used to plug in and recharge battery pack 8. A rocker switch 5 can be used to turn motor 9 on and off. A control system 7 can be used to control operation of motor 9. In preferred embodiments, control system 7 can be used to adjust the speed of motor 9 or even to vary the speed over time (in addition to merely turning the device on or off).

In preferred illustrated embodiments, there are two moving arms 1 (positioned at 180 degrees apart to one another on axle 12). It is to be understood that the present invention is not so limited. For example, the present system also encompasses configurations with three or more arms 1. (For example, three rotating arms 1 can be positioned at 120 degrees to one another on axle 12).

In further optional embodiments, a heater can be disposed in housing 25 for delivering therapeutic heat to the user; and/or a vibrating element can also be disposed in housing 25 for delivering therapeutic vibration to the user. For example, heating can be accomplished by using mini infra-red lightbulbs in the interior of the housing, around the perimeter of flexible membrane 22. It is to be understood, however, that other heating methods can be used instead, all keeping within the scope of the present invention. In addition, vibration can be added by any system that spins an imbalanced weight with a motor. It is to be understood, however, that other vibrating system and methods can be used instead, all keeping within the scope of the present invention.

In preferred aspects, the present system also provides a method of improving gastric reflux, treating hiatal hernias and reducing abdominal tension by: placing a flexible wall membrane 22 of an abdominal massaging device 30 against a user's chest immediately below the user's sternum, and then rotating the massage arms 1 in housing 25 such that arms 1 sequentially push against flexible wall membrane 22, thereby moving in a downward direction D against the user's chest. This optional method preferably further comprises positioning a pair of downwardly, outwardly tapering top edges 27 and 28 of housing 25 against the bottom of the user's rib cage below the user's sternum such that edges 27 and 28 push against the bottom of the user's rib cage and sternum as rotating arms 1 moving in the downwardly direction D to prevent the housing 25 from “walking up” the patient's chest.

In operation, the movement of the sequential rotating arms 1 pushing downwardly just below the xiphoid process of the sternum in the center of the upper abdomen provides a relaxing, rotary massaging motion. This action relaxes the diaphragm, allows the stomach to move inferiorly and stimulates parasympathetic nerves to improve digestion, thereby relaxing the abdomen and enhancing proper diaphragmatic breathing.

What is claimed is:

1. An abdominal massaging assembly, comprising:
a housing having an internal cavity therein;
a flexible wall membrane disposed on one side of the housing;
an elastic support member connected to the housing for supporting the housing such that the flexible wall membrane is positioned against a user's chest immediately below the user's sternum; and
a plurality of rotating massage arms disposed within the internal cavity in the housing, wherein the rotating arms sequentially push the flexible wall membrane against the user's chest, and wherein the rotating arms sequentially move in a downwardly direction against the flexible wall membrane.

2. The assembly of claim 1, wherein the top of the housing comprises a pair of downwardly, outwardly tapering edges dimensioned to rest against the bottom of the user's rib cage at the bottom of the user's sternum.

3. The assembly of claim 2, wherein the downwardly, outwardly tapering edges of the housing push against the bottom of the user's rib cage and sternum as the rotating arms moving in the downwardly direction against the flexible wall membrane push the housing upwards on the user's chest.

4. The assembly of claim 1, wherein each of the plurality of rotating massage arms has a roller at its distal end, and wherein the roller rolls against an interior surface of the flexible wall membrane.

5. The assembly of claim 1, wherein the plurality of rotating massage arms extend from a rotating axle mounted within the internal cavity in the housing.

6. The assembly of claim 5, wherein the rotating axle extends in a direction parallel to the elastic support member.

7. The assembly of claim 1, further comprising:
a motor disposed in the housing for rotating the plurality of arms;
a battery for powering the motor to rotate the arms; and
a control system for controlling operation of the motor.

8. The assembly of claim 7, wherein the control system adjusts the speed of the motor.

9. The assembly of claim 1, wherein the housing has a generally triangular shape.

10. The assembly of claim 1, wherein each of the rotating arms sequentially travels in a downwards path along the user's chest.

11. The assembly of claim 1, wherein the plurality of arms comprises two arms.

12. The assembly of claim 1, wherein the plurality of arms comprises three arms.

13. The assembly of claim 1, further comprising:
a heater disposed in the cavity for delivering therapeutic heat to the user.

14. The assembly of claim 1, further comprising:
a vibrating element disposed in the cavity for delivering therapeutic vibration to the user.

15. The assembly of claim 1, wherein the elastic support member wraps around the torso of the user.

16. A method of improving gastric reflux, treating hiatal hernias and reducing abdominal tension, comprising:
placing a flexible wall membrane of an abdominal massaging device against a user's chest immediately below the user's sternum, the abdominal massaging device comprising:
a housing with the flexible wall membrane disposed on one side of the housing, and
a plurality of rotating massage arms disposed within the housing, wherein rotation of the massage arms pushes the flexible wall membrane against the user's chest; and
rotating the massage arms in the housing such that the arms sequentially push against the flexible wall membrane in a downwards direction against the user's chest.

17. The method of claim 16, further comprising:
positioning a pair of downwardly, outwardly tapering top edges of the housing against the bottom of the user's rib cage below the user's sternum such that the downwardly, outwardly tapering edges of the housing push up against the bottom of the user's rib cage and sternum as the rotating arms moving in the downwardly direction against the flexible wall membrane push the housing upwards on the user's chest.

18. The method of claim 16, further comprising:
supporting the abdominal massaging device against the user's chest with an elastic support member connected to the housing wrapping around the torso of the user.

19. The method of claim 16, wherein each of the plurality of rotating massage arms has a roller at its distal end, and wherein the roller rolls against an inside surface of the flexible wall membrane.

20. The method of claim 16, further comprising:
controlling the speed of the rotating arms with a control system in the abdominal massaging device.

21. The method of claim 16, further comprising:
delivering therapeutic heat to the user with a heater disposed in the abdominal massaging device; or
delivering therapeutic vibration to the user with a vibrating element disposed in the abdominal massaging device.
